(19) United States
(12) Patent Application Publication
(10) Pub. No.: US 2025/0325233 A1
(43) Pub. Date: Oct. 23, 2025

(54) DEVICES AND METHODS OF DIAGNOSING MEDICAL CONDITIONS

(71) Applicant: ARDIS LTD., Katzrin (IL)

(72) Inventors: Alexander MELAMUD, Haifa (IL); Yehuda RINGEL, Tel-Aviv (IL); Arkady GLUKHOVSKY, Nesher (IL); Eyal AVIRAM, Herzliya (IL)

(21) Appl. No.: 18/861,346

(22) PCT Filed: May 4, 2023
(86) PCT No.: PCT/IL2023/050459

§ 371 Date: Oct. 29, 2024

Related U.S. Application Data
(60) Provisional application No. 63/338,283, filed on May 4, 2022.
(60) Provisional application No. 63/411,122, filed on Sep. 29, 2022.

(51) Int. Cl. A61B 5/00; A61B 5/03; A61M 25/00; A61M 25/10

(52) U.S. Cl. CPC A61B 5/6853; A61B 5/036; A61B 5/4255; A61B 5/4337; A61M 2025/0002; A61M 25/10184; A61M 2205/3306

(57) ABSTRACT

Medical testing devices and methods of diagnosing are disclosed. One device may include, a catheter; an inflatable balloon located in a distal portion of the catheter, wherein the inflatable balloon is at least partially insertable into a body cavity; one or more pressure sensors located inside the inflatable balloon; an inflating unit in fluid connection with the inflatable balloon; and a control unit attached to the catheter and configured to receive pressure measurements from the one or more pressure sensors; and to control the inflating unit to control the pressure inside the inflatable balloon based on the received measurements.

### CROSS-REFERENCE TO RELATED APPLICATIONS

This application claims the benefit of priority to U.S. Provisional Application No. 63/338,283, titled “COMPREHENSIVE ANORECTAL DIAGNOSTIC (CARD) SYSTEM”, filed on May 4, 2022 and U.S. Provisional Application No. 63/411,122, titled “COMPREHENSIVE ANORECTAL DIAGNOSTIC (CARD) SYSTEM”, filed on Sep. 29, 2022, the contents of which are incorporated herein by reference in their entirety.

### FIELD OF THE INVENTION

The present invention relates generally to devices and methods for diagnosing medical conditions. More specifically, the present invention relates to devices and methods for diagnosing medical conditions in a body cavity, such as the rectum.

### BACKGROUND OF THE INVENTION

Medical conditions related to disorders or dysfunction of the human pelvic floor are hard to accurately diagnose. For example, diagnosing disorders or dysfunction of the rectal and anorectal functions requires accurate measurements for understanding individual patient's anorectal sensorimotor dysfunction and directing personalized and effective treatment in patients suffering from pelvic floor disorders.

Pelvic floor disorders include motor/motility problems, such as emptying abnormalities of the lower gastrointestinal and urinary tracts, fecal and/or urinary incontinence; anatomical problems such as pelvic organ prolapse, rectocele, and enterocele; and sensory abnormalities of the lower gastrointestinal and urinary tracts. These disorders are widely prevalent, often not diagnosed or not accurately diagnosed, and are largely undertreated.

Different methodologies are used in the gastroenterology clinical practice to evaluate patients with these disorders, including anorectal manometry, defecography, pelvic MRI, neurophysiological testing, and the like.

The currently used device for anorectal manometry, is illustrated in FIG. 1, shows an anorectal device 50 insertable into the rectum. Anorectal device 50 comprises an anorectal catheter 10 and an inflatable balloon 20 located in a distal portion of catheter 10. Device 50 further includes at least one rectal pressure sensor 30 configured to measure the pressure inside the balloon and an array of anal pressure sensors 40. Pressure sensors 30 and 40 are embedded or attached to catheter 10. Balloon 20 is inflated after introduction into the rectal space. Balloon 20 is inflated with air or other gases, or it can be inflated with a liquid, such as mineral oil. In currently used methods a constant volume of gas or liquid (e.g., 50 ml) is inserted during each test. Pressure applied by the rectal walls to balloon 20 is measured by pressure sensor 30. Pressure data from the array of pressure sensors can be transmitted either wire(s) or wirelessly to the external processing module 40, which can display and/or analyze the pressure profile developed by the rectal walls.

Anorectal manometry is conducted while the patient is in a left lateral position at the patient is laying on his/her side while bending his/her knees. Other tests like during dynamic pelvic floor MRI, are conducted in the supine position when the patient is laying on the back. Both positions have significant limitations that prevent a complete and actual understanding of the patient's problem and thus direct personalized and effective treatment. As both positions are non-seated positions, they are limited and may often be misleading in the assessment of anorectal junction descent and changes in anorectal angle, two important parameters in the assessment of defecation and bowel incontinence.

Furthermore, all currently available methods must be conducted in a clinic or a hospital by a professional.

Therefore, there is a need for an accurate testing device and method that can be conducted in a seated position, for example, by a user at home.

### SUMMARY OF THE INVENTION

Some aspects of the invention are directed to a device, comprising: a catheter; an inflatable balloon located in a distal portion of the catheter, wherein the inflatable balloon is at least partially insertable into a body cavity; one or more pressure sensors located inside the inflatable balloon; an inflating unit in fluid connection with the inflatable balloon; and a control unit attached to the catheter and configured to receive pressure measurements from the one or more pressure sensors; and to control the inflating unit to control the pressure inside the inflatable balloon based on the received measurements.

In some embodiments, at least one of said one or more pressure sensors is attached to a surface of said inflatable balloon. In some embodiments, at least one of said one or more pressure sensors is attached to the catheter. In some embodiments, the catheter is an anorectal catheter. In some embodiments, said one or more pressure sensors are selected from: polyvinylidene fluoride (PVDF) membrane, piezoresistive strain gauges, capacitive sensors, electromagnetic sensors, piezoelectric sensors, and optical sensors.

In some embodiments, said inflatable balloon comprises two or more portions separately inflatable by a separate inlet, and wherein one or more pressure sensors are attached to a surface of each portion. In some embodiments, said control unit comprises a communication module configured to send said pressure measurements to a remote computing device. In some embodiments, the communication module is further configured to receive instruction to control the pressure inside the inflatable balloon, from the remote computing device. In some embodiments, the device further comprises one or more additional pressure sensors attached to a portion of the catheter that is external to the balloon. In some embodiments, the pressure inside said inflatable balloon is further controlled based on measurements from the one or more additional pressure sensors.

In some embodiments, the device further comprises one or more image sensors located inside the balloon. In some embodiments, the one or more image sensors are attached in proximity to a distal end of said catheter. In some embodiments, said one or more image sensors are located at least in one of: in proximity to a distal end of the catheter and inside said catheter at a distance from said distal end. In some embodiments, the one or more image sensors are attached to an inner surface of said inflatable balloon. In some embodiments, at least one of said image sensors is configured to capture images of the internal shape of said inflatable balloon.

In some embodiments, the device further comprises one or more optical reflectors located inside said inflatable balloon, said one or more optical reflectors are configured to deploy upon command from said controller. In some embodiments, the device further comprises one or more electrodes attached on an outer surface of said inflatable balloon and configured to deliver electrical signals to rectal muscles. In some embodiments, a location of one or more electrodes is determined such that in operation said one or more electrodes are in contact with the rectal muscles upon the inflation of the balloon.

In some embodiments, the device further comprises one or more motion detection sensors attached to said catheter configured to detect a motion of the catheter.

Some additional aspects of the invention are directed to a method of diagnosing an anorectal condition, comprising: receiving, optionally in real-time, pressure measurements from one or more pressure sensors located inside an inflatable balloon attached to a catheter, when the catheter is inserted to the rectum; controlling in real time the pressure inside said inflatable balloon based on the pressure; identifying in said pressure measurements, associated with pelvic floor disorder; and diagnosing the anorectal condition based on the identified measurements.

In some embodiments, controlling the pressure inside said inflatable balloon, in real time, comprises inflating the inflatable balloon until a pressure associated with a sensation of urge for defecation is reached. In some embodiments, said measurements are associated with pelvic floor disorder, measurements associated with a sensation of urge for defecation, anal pressure level, rectal pressure level, and a difference between rectal and anal pressures.

In some embodiments, receiving the pressure measurements comprises receiving a plurality of pressure measurements each associated with a different section of said inflatable balloon, and wherein each section forms a separate balloon volume.

In some embodiments, diagnosing the anorectal condition comprises creating a 3D pressure map inside the balloon based on the received plurality of pressure measurements. In some embodiments, the method, further comprises: receiving from at least one image sensor located inside said inflatable balloon one or more images of internal surfaces of said inflatable balloon; and wherein diagnosing the anorectal condition is further based on the received images. In some embodiments, diagnosing comprises constructing a 3D model of the inflatable balloon based on the images, wherein the 3D model is indicative of the pressure profile applied to the balloon by rectal muscles.

In some embodiments, the images are received from at least two different angles inside said inflatable balloon. In some embodiments, the images are received from at least two different image sensors located inside said inflatable balloon. In some embodiments, the anorectal condition is a rectocele condition and diagnosing comprises detecting distortion in the 3D model indicative of budging and herniation of the rectum into the back wall of the vagina.

In some embodiments, the method further comprises: calibrating said one or more image sensors using a calibration pattern located inside the balloon. In some embodiments, the method further comprises controlling an array of electrodes located on an outer surface of the balloon to provide electrical stimulation to the rectal muscles. In some embodiments, the method further comprises: receiving, from one or more motion sensors attached to said catheter, motion signals indicative of the movement of said catheter inside the rectum; and determining an effectiveness of contraction of the rectal muscles based on the received motion signals.

Some additional aspects of the invention are directed to an additional device, comprising: a catheter; an inflatable balloon located in a distal portion of the catheter, wherein the inflatable balloon is at least partially insertable into a body cavity; one or more image sensors located inside the inflatable balloon; an inflating unit in fluid connection with the inflatable balloon; and a control unit attached to the catheter and configured to receive images of the internal shape of said inflatable balloon from the one or more image sensors; and to control the inflating unit to control the pressure inside the inflatable balloon based on the received images.

In some embodiments, the one or more image sensors are attached in proximity to a distal end of said catheter. In some embodiments, at least one of said one or more image sensors is located on the catheter inside said balloon at a distance from said distal end. In some embodiments, at least one of the one or more image sensors is attached to an inner surface of said inflatable balloon. In some embodiments, the device further comprises one or more optical reflectors located inside said inflatable balloon, said one or more optical reflectors are configured to deploy upon command from said control unit.

In some embodiments, the device further comprises one or more electrodes attached on an outer surface of said inflatable balloon and configured to deliver electrical signals to rectal muscles. In some embodiments, a location of one or more electrodes is determined such that said one or more electrodes are in contact with the rectal muscles upon inflation of the balloon.

In some embodiments, the device further comprises one or more motion sensors.

Some additional aspects of the invention may be directed to a method of diagnosing anorectal condition, comprising: receiving, optionally in real-time, images from one or more image sensors located inside an inflatable balloon attached to a catheter, when the catheter is inserted to the rectum; controlling in real time the pressure inside said inflatable balloon based on the images of internal surfaces of said inflatable balloon; identifying in said image the shape of the balloon, associated with a pelvic floor disorder; and diagnosing the anorectal condition based on the identified images.

Some additional aspects of the invention are directed to an additional device, comprising: a segmented catheter comprising two or more sealed hollow segments, wherein at least some of the sealed segments comprise at least one pressure sensor and is connected to a neighboring segment via a flexible connector; one or more motion sensors configured to measure a relative angle between at least two segments; and a control unit attached to the catheter and configured to: receive pressure measurements from at least two pressure sensors and the relative angle between at least two segments comprising the at least two pressure sensors; diagnose a medical condition based on the received pressure measurements and the corresponding relative angle.

In some embodiments, the one or more motion sensors are attached to different segments. In some embodiments, the one or more motion sensors are accelerometers.

In some embodiments, each sealed hollow segment is filled with a fluid, selected from, liquid, gas, and gel. In some embodiments, a first sealed hollow segment is filled with a first type of fluid and a second hollow segment is filled with a second type of fluid, different from the first. In some embodiments, the at least one pressure sensor is selected from: polyvinylidene fluoride (PVDF) membrane, piezoresistive strain gauges, capacitive sensors, electromagnetic sensors, piezoelectric sensors, and optical sensors. In some embodiments, at least one segment comprises an inflatable balloon.

In some embodiments, the device further comprises a flexible cover covering the two or more sealed hollow segments, while providing a flexible connection between the two or more sealed hollow segments.

Some additional aspects of the invention are directed to an additional method of diagnosing anorectal condition, comprising: receiving, optionally in real-time, pressure measurements from two or more pressure sensors each is attached to a segment of a segmented anorectal catheter, when the catheter is inserted to the rectum; receiving motion measurements from motion sensors attached to at least two different segments of the segmented anorectal catheter; calculating a relative angle between the at least two different segments based on the motion measurements; and diagnosing a medical condition based on the received pressure measurements and a corresponding relative angle.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of a prior art anorectal device for an anorectal manometry test.

FIG. 2 is an illustration of a testing device comprising pressure sensors according to some embodiments of the invention;

FIGS. 3A and 3B are illustrations of the positions of the testing device inside the rectum according to some embodiments of the invention;

FIGS. 4A and 4B are illustrations of another testing device comprising pressure sensors according to some embodiments of the invention;

FIG. 5 is an illustration of a testing device comprising image sensors according to some embodiments of the invention;

FIG. 6 is an illustration of another testing device comprising image sensors according to some embodiments of the invention;

FIG. 7 is an illustration of a testing device comprising image sensors and reflectors according to some embodiments of the invention;

FIG. 8 is an illustration of a calibration array of markers for calibrating a testing device comprising image sensors according to some embodiments of the invention;

FIG. 9 is an illustration of another testing device comprising image sensors according to some embodiments of the invention;

FIGS. 10A, 10B, 10C, and 10D are illustrations testing devices comprising pressure sensors and/or motion sensors according to some embodiments of the invention;

FIG. 11 is an illustration of a testing device comprising image sensors, pressure sensors, and motion sensors according to some embodiments of the invention;

FIG. 12 is an illustration of another testing device comprising image sensors, pressure sensors, and motion sensors according to some embodiments of the invention;

FIGS. 13A and 13B are a graph and an illustration of measurements of pressure and angle taken using the device of FIG. 10A, according to some embodiments of the invention;

FIGS. 14A, 14B, 14C, and 14D show orientation (relative angles) between segments of a catheter in a device when inserted into the anal canal and the rectum according to some embodiments of the invention;

FIG. 15 is a flowchart of a method of diagnosing anorectal condition according to some embodiments of the invention;

FIG. 16 is a flowchart of another method of diagnosing anorectal condition according to some embodiments of the invention;

FIG. 17 is a flowchart of yet another method of diagnosing anorectal condition according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of the same or similar features or elements may not be repeated.

Embodiments of the invention are directed to a testing device insertable into a body cavity, such as, the rectum, and can measure directly or indirectly pressure applied by muscles of the body cavity (e.g., rectal muscles) on the device. Measuring the pressure may provide an accurate diagnosis of medical conditions, such as, pelvic floor disorders.

A testing device according to embodiments of the invention can be used in a seated position, left lateral position, or supine position. In some embodiments, the testing device may simply be operated by the user (e.g., the patient). The device may include a local control unit that can communicate with a computing device associated with a professional/a clinic/hospital for providing data that can be used for diagnosis and selection of treatment.

The testing device according to embodiments of the invention can be also suitable for use in medical conditions other than chronic constipation and fecal incontinence, for example, in veterinary medicine, especially for the diagnostics of different GI Diseases in Animals, specifically in cats, dogs, horses, camels, cows, pigs, and others.

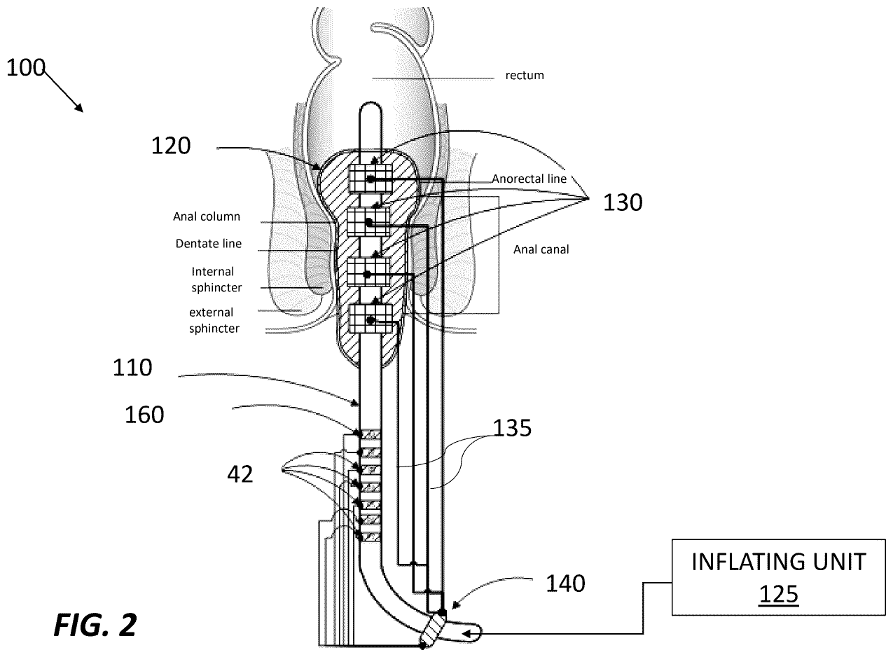

Reference is now made to FIG. 2 which is an illustration of a device according to some embodiments of the invention. In the nonlimiting example, illustrated in FIG. 2, a device 100 is configured to be used for diagnosing chronic constipation and fecal incontinence in humans. However, as should be appreciated by one skilled in the art, device 100 can be used for other purposes, to be inserted into other body cavities.

Device 100 may include a catheter 110 and an inflatable balloon 120 located in a distal portion of catheter 110, wherein inflatable balloon 120 is at least partially insertable into a body cavity. For example, catheter 110 may be an anorectal catheter and the body cavity may be the rectum, as illustrated in FIG. 2.

Catheter 110 may be flexible and hollow and may be configured to hold communication and power cables inserted therethrough (not illustrated) for electrically connecting and/or communicating various components of device 100. As used herein, the term flexible may refer to a property of the catheter that allows the catheter to bend when inserted into the body cavity, thus following the internal curvature of the body cavity.

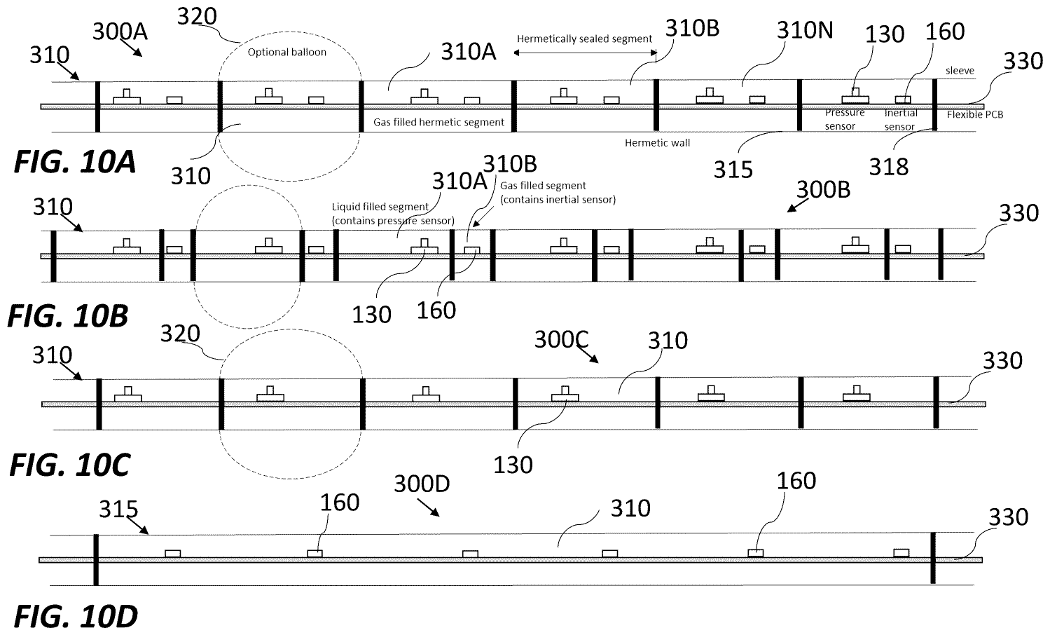

In some embodiments, catheter 100 may be a segmented catheter, as disclosed and discussed with respect to devices 300A, 300B, 300C and 300D illustrated in FIGS. 10A-10D.

Inflatable balloon 120 may be in fluid connection with an inflating unit 125 that may be, fully or partially located externally to the body cavity. Inflation fluid may be introduced into inflatable balloon 120 via an internal lumen in catheter 110 (not illustrated). Nonlimiting examples of inflation fluids may include, air, water, oil, gel, and the like.

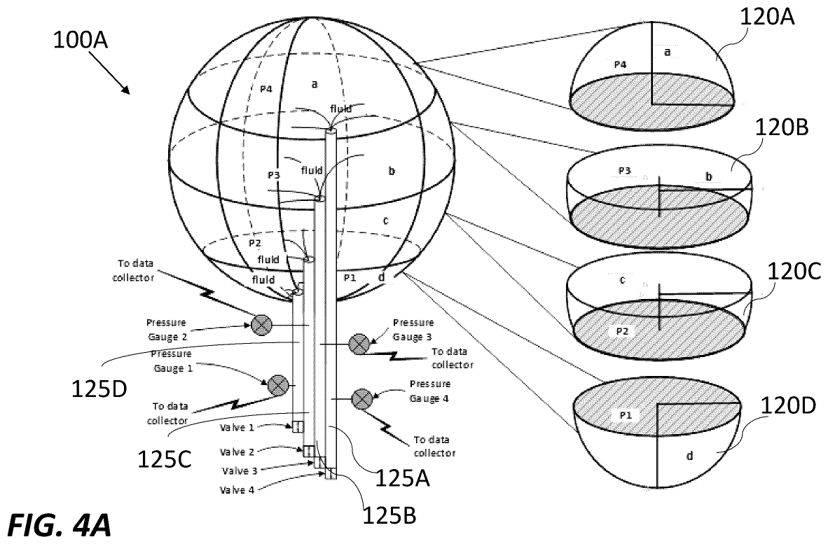

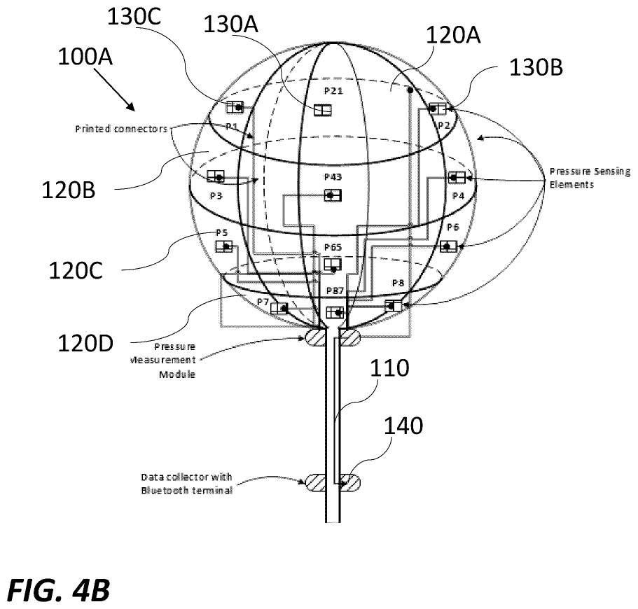

Device 100 may further include one or more pressure sensors 130 located inside inflatable balloon 120. For example, one or more pressure sensors 130 are attached to a surface of said inflatable balloon, as illustrated also in FIG. 4B. In some embodiments, one or more pressure sensors 130 may be attached to the inner surface and/or the outer surface of balloon 120. Additionally or alternatively, one or more pressure sensors 130 may be attached to catheter 110. In some embodiments, one or more pressure sensors 130 may be selected from: polyvinylidene fluoride (PVDF) membrane, piezoresistive strain gauges, capacitive sensors, electromagnetic sensors, piezoelectric sensors, optical sensors and the like.

As used herein, the term ‘attached’ may refer to any physical connection between one element to another. For example, elements can be permanently attached (e.g., fixed, glued, welded, etc.) or detachably connected via a connector (e.g., a clip, a sticker, and the like).

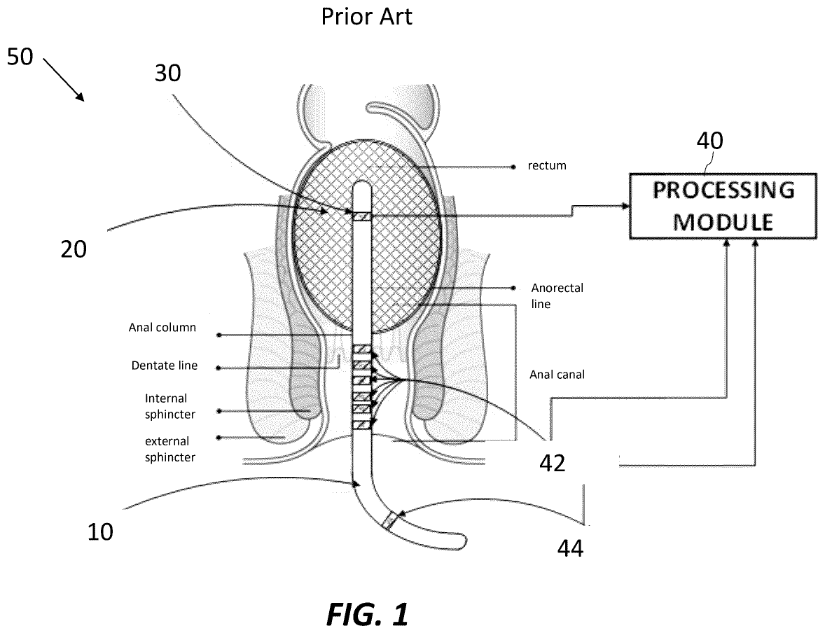

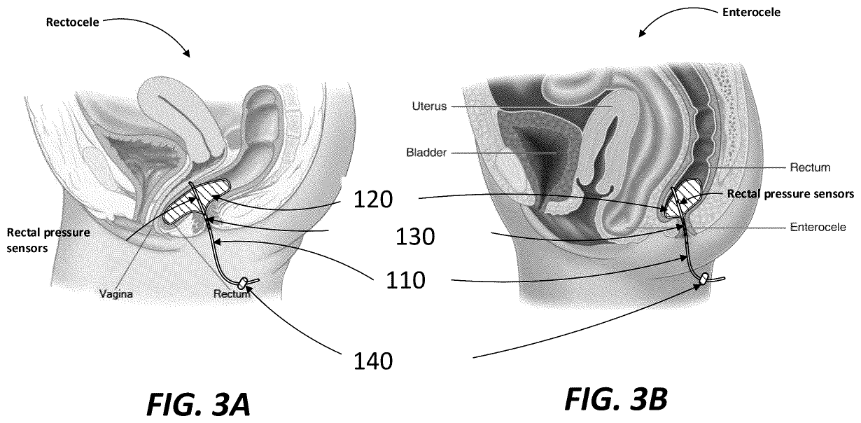

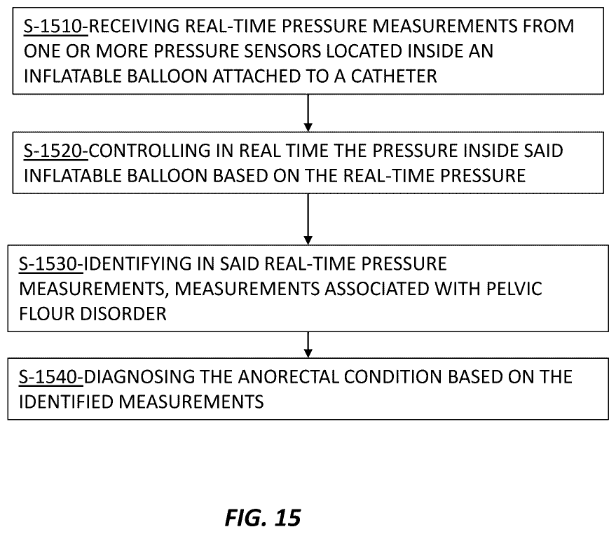

Reference is now made to FIGS. 3A and 3B which show device 100 with an inflatable balloon 120 inserted into a rectal space during a diagnostic test. Unlike the current practice, illustrated in FIG. 1, in which balloon 20 expulsion test is done following routine inflation of the intra-rectal balloon with 50 ml water/air for all patients, device 100 enables inflating the balloon to the pressure inducing a sensation of urge for defecation of each individual patient. Thus eliminating differences in rectal size and/or compliance and enabling personalized and more accurate assessment of defecation and continence physiological functions, as discussed with respect to the method illustrated in the flowchart of FIG. 15.

In some embodiments, inflatable balloon 120 is separated into two or more portions, as illustrated in FIGS. 4A and 4B. Device 100 may include inflatable balloon 120 separated into two or more portions (e.g., 4 portions) 120A, 120B, 120C, and 120D each being inflated through a separate inlet 125A, 125B, 125C, and 125D included in inflating unit 125. In some embodiments, all separate inlets 125A, 125B, 125C, and 125D may be fed from a single feeding line, or each may be fed by a separate feeding line, as illustrated. In some embodiments, device 100A may further include pressure gauges (e.g., pressure gauges 1, 2, 3, and 4 illustrated in FIG. 4A) for measuring the pressure of the fluid provision for each portion 120A, 120B, 120C, and 120D and valves (e.g., valves 1, 2, 3, and 4 illustrated in FIG. 4A) for controlling the provision of fluid into the feeding lines.

In some embodiments, one or more pressure sensors 130A, 130B, and 130C are attached to at least one surface of each portion, for example, portion 120A, as illustrated in FIG. 4B.

In some embodiments, inflatable balloon 120 may include internal separations made from a flexible material that can be similar or different from the material of balloon 120. In some embodiments, all connection lines between balloon 120 and the separation of portions 120A, 120B, 120C and 120D may be sealed for the passage of the fluid. Therefore, one or more pressure sensors 130 attached/included in each portion may measure the pressure form this portion only.

Referring back to FIG. 2, in some embodiments, devices 100 and 100A may further include a control unit 140 attached to catheter 110 and configured to receive pressure measurements from one or more pressure sensors 130, 130A-130D; and to control inflating unit 125 to control the pressure inside inflatable balloon 120 and 120A-120D based on the received measurements. In some embodiments, control unit 140 may comprise a communication module (not illustrated) configured to send the pressure measurements to a remote computing device, for example, device 40 illustrated in FIG. 1. In some embodiments, the communication module is further configured to receive instruction to control the pressure inside the inflatable balloon, from the remote computing device. A nonlimiting example of a method of controlling devices 100 and 100A and the use of these measurements for diagnosing a medical condition is given with respect to the flowchart of FIG. 15.

In some embodiments, devices 100 and 100A may further include one or more additional pressure sensors 42 attached to catheter 110 external to balloon 120. For example, pressure sensors 42 may be configured to measure the pressure in the anal canal. In some embodiments, the pressure inside said inflatable balloon is further controlled based on measurements from the one or more additional pressure sensors.

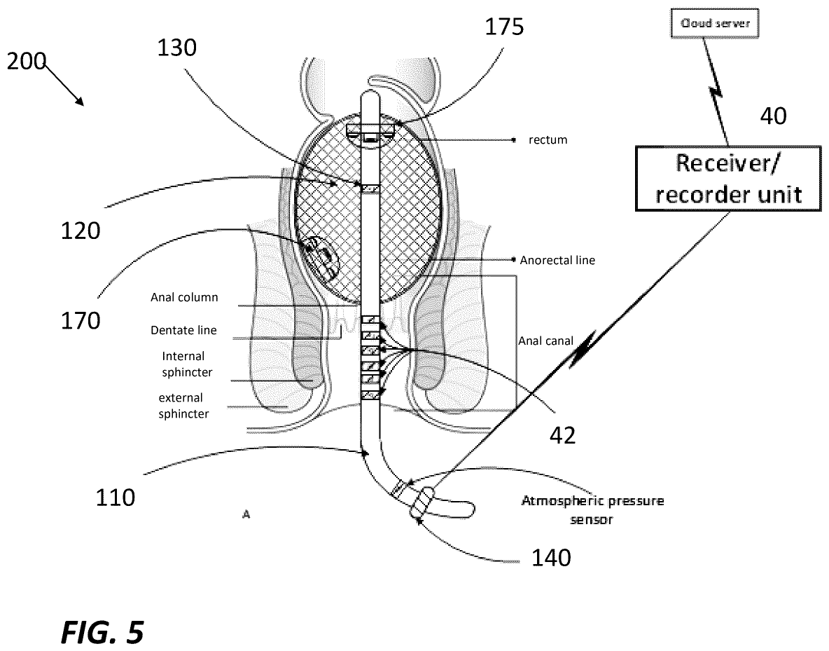

In some embodiments, devices 100 and 100A may further include one or more image sensors 170 and 175 located inside the balloon, as illustrated and discussed with respect to the embodiments of FIGS. 5, 6, 7, 8, and 9.

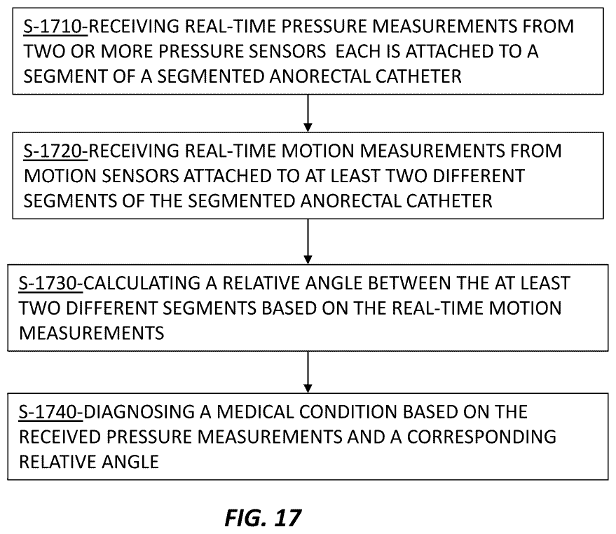

In some embodiments, devices 100 and 100A may further include one or more motion sensors 160 attached to catheter 110. Motion sensors 160 may include any sensor configured to detect movements, for example, accelerometers. In some embodiments, control unit 140 may be configured to receive signals from one or more motion sensors 160 and to use the signals for diagnosing medical conditions. A detailed discussion is given with respect to FIGS. 10A to 10D and the method of FIG. 17.

Reference is now made to FIG. 5 which is an illustration of a device (e.g., a testing device) according to some embodiments of the invention. In a non-limiting example illustrated in FIG. 5, a device 200 may be configured to be used for diagnosing chronic constipation and fecal incontinence in humans. However, as should be appreciated by one skilled in the art, device 200 can be used for other purposes, to be inserted into other body cavities.

Device 200 may include a catheter 110 and an inflatable balloon 120 located in a distal portion of catheter 110, wherein inflatable balloon 120 is at least partially insertable into a body cavity. For example, catheter 110 may be an anorectal catheter and the body cavity may be the rectum.

Catheter 110 may be flexible and hollow and may be configured to hold communication and power cables inserted therethrough (not illustrated) for electrically connecting and/or communicating various components of device 200. In some embodiments, catheter 110 may be a segmented catheter, as disclosed and discussed with respect to devices 300A, 300B, 300C, and 300D illustrated in FIGS. 10A-10D.

Inflatable balloon 120 may be in fluid connection with an inflating unit (e.g., unit 125 illustrated in FIG. 2), that may be, fully or partially, located externally to the body cavity. Inflation fluid may be introduced into inflatable balloon 120 via an internal lumen in catheter 110 (not illustrated). Nonlimiting examples of inflation fluids may include, air, water, oil, gel, and the like.

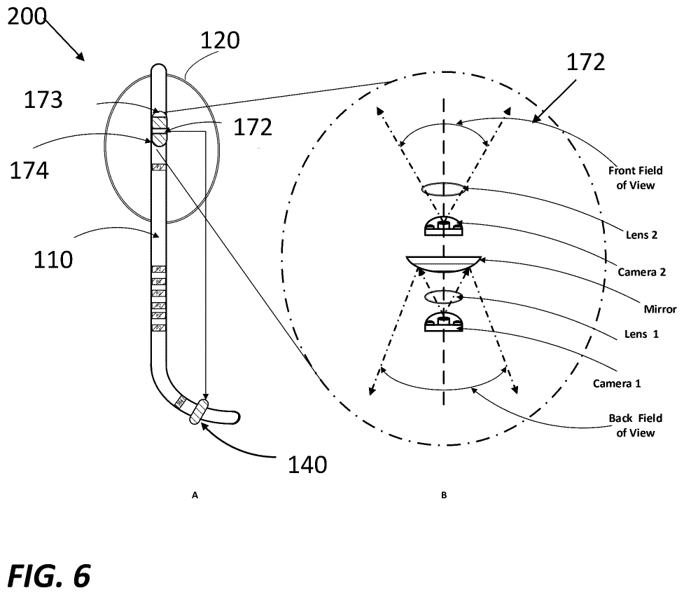

In some embodiments, device 200 may further include one or more image sensors 170 and 175 located inside inflatable balloon 120. For example, one or more image sensors 170 may be attached in proximity to a distal end of catheter 110 and/or located on catheter 110 at a distance from the distal end, as illustrated. In yet another example, one or more image sensors 175 are attached to the inner surface of inflatable balloon 120. As should be understood by one skilled in the art the locations of image sensors 170 and 175 illustrated in FIGS. 6-9 are given as examples only and the invention is not limited to these specific locations.

Some nonlimiting examples for image sensors 170 and 175 may include cameras (e.g., charge-coupled device (CCD) or complementary metal-oxide semiconductor (CMOS) cameras, etc.), light-emitting diode (LED) based image sensors/cameras and the like. In some embodiments, image sensors 170 and 175 may be connected to catheter 110 and/or control unit 140 via either wired or wireless communication.

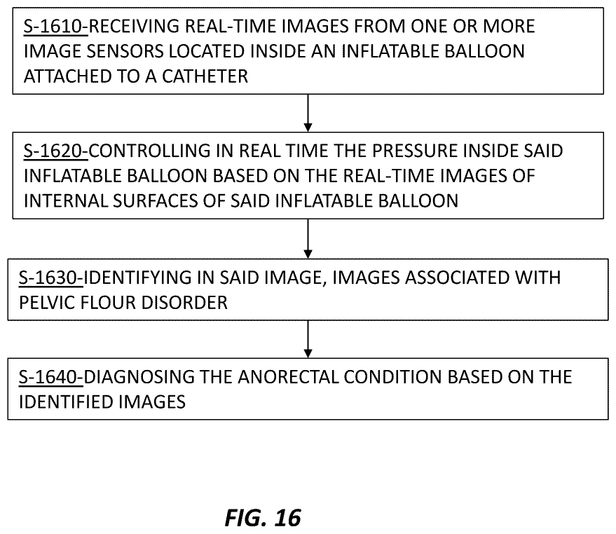

In some embodiments, device 200 may include control unit 140 attached to catheter 110 and configured to receive images of the internal shape of inflatable balloon 120 from one or more image sensors 170 and 175; and to control the inflating unit to control the pressure inside inflatable balloon 120 based on the received images. In some embodiments, analysis of the images acquired by the image sensors of the inner surface of the balloon may enable to recreate the shape of the balloon, and thus recreate the pressure profile applied to the balloon by the body cavity, for example, the rectal muscles. In some embodiments, once the pressure profile of the muscle is applied to the balloon, the shape of the balloon is distorted, and the geometrical pattern visible by the cameras is distorted as well. Measuring the difference between the distorted pattern and the known original pattern may allow recreating the shape of the balloon, and thus also recreating the profile of the pressure applied by the muscles (e.g., the rectal muscles). A nonlimiting method for using these images for controlling device 200 and/or diagnosing a medical condition is given with respect to the flowchart of FIG. 16.

In some embodiments, control unit 140 may comprise a communication module (not illustrated) configured to send the images to a remote computing device, for example, remote computing device 40, which can be a cloud server.

In some embodiments, device 200 may further include one or more pressure sensors 130 located inside inflatable balloon 120. For example, one or more pressure sensors 130 are attached to the surface of said inflatable balloon, as illustrated also in FIG. 4B. In some embodiments, one or more pressure sensors 130 may be attached to inner surface and/or the outer surface of balloon 120. Additionally or alternatively, one or more pressure sensors 130 may be attached to catheter 110. In some embodiments, one or more pressure sensors 130 may be selected from: polyvinylidene fluoride (PVDF) membrane, piezoresistive strain gauges, capacitive sensors, electromagnetic sensors, piezoelectric sensors, optical sensors and the like.

Device 200 may further include one or more additional pressure sensors 42 attached to catheter 110 external to balloon 120. For example, pressure sensors 42 may be configured to measure the pressure in the anal canal. In some embodiments, the pressure inside said inflatable balloon is further controlled based on measurements from the one or more additional pressure sensors.

Reference is now made to FIG. 6 which is an illustration of another device (e.g., a testing device) according to some embodiments of the invention. Device 200 of FIG. 6 may include substantially the same components and elements of device 200 of FIG. 5. In some embodiments, device 200 may include image sensor 172 having an optical design enabling a video camera to see most of the internal surface of inflatable balloon 120. Two cameras 173 and 174 are located along the axis of catheter 110. The Distal (front) camera 173 shown provides a forward-looking field of view (FOV). The Proximal (back) camera 174 uses a convex mirror to provide backward-looking FOV. Combining FOVs of front and back cameras 173 and 174 allows for imaging of most of the internal surface of balloon 120.

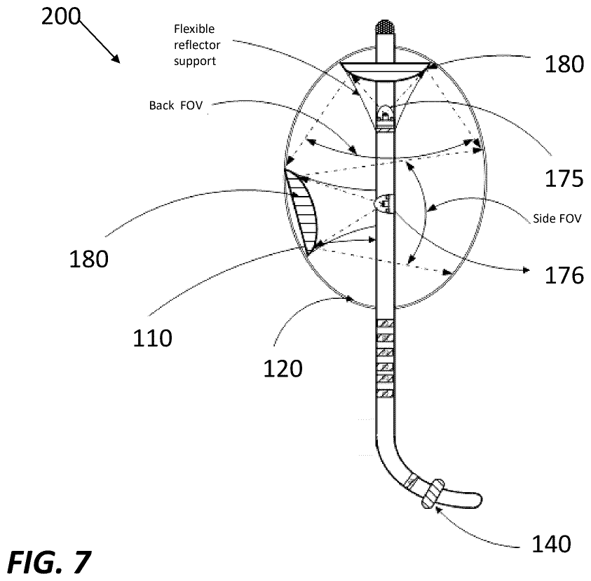

Reference is now made to FIG. 7 which is an illustration of another device 200 according to some embodiments of the invention. Device 200 of FIG. 7 may include substantially the same components and elements of device 200 of FIG. 5 and/or FIG. 6. In some embodiments, device 200 may further include one or more optical reflectors 180 located inside inflatable balloon 120. In some embodiments, one or more optical reflectors 180 are configured to deploy upon command from control unit 140. Each one of optical reflectors 180 may be located in front of the FOV of a corresponding image sensor 170 or 175. Each one of optical reflectors 180 may include a support and a deployment mechanism.

In some embodiments, in order to improve the outcome of the image processing algorithm, the inner wall of the balloon may be marked with a pre-defined geometric pattern, such as a rectangular grid or other geometrical patterns. It should be appreciated by those skilled in the art that the deviation of the captured geometrical patterns from the expected (actual) patterns may be indicative of the deformation of the surface, the distance of each part of the marked surface from the image sensor, and the relative location and orientation of the imager with respect to each portion of the geometrical pattern captured by the imager.

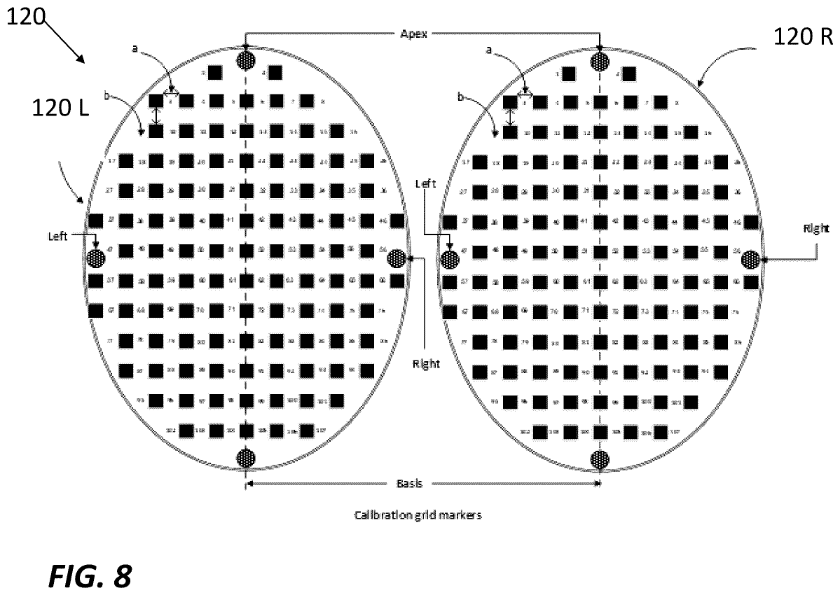

Reference is now made to FIG. 8 which is an illustration of a calibration array for image sensors included in device 200 according to some embodiments of the invention. In some embodiments, balloon's 120 internal surface may be at least partially covered with optical calibration patterns 120L and 120R. Optical calibration patterns 120L and 120R may include a pattern of similar or different geometrical shapes. Some nonlimiting examples may include, dots, rectangular grids, and/or patterns of dots located on rectangular, triangular, or other patterns. Therefore, the calibration pattern may allow capturing only a portion of the inner surface of balloon 120 and using the known locations of the geometrical shapes to extrapolate the entire inner shape of balloon 120.

In some embodiments, when inflated in the rectum, the shape of the balloon may be distorted according to the inner shape of the rectum. Since the human rectum does not have sharp angles, the shape of the balloon is expected to be smooth, not having sharp angles either. Under these conditions, the distortion of the balloon pattern will be smooth, and it may be possible to interpolate the location of the obscured part of the calibration pattern (e.g., pattern 120L and/or pattern 120R). For example, it is possible to apply linear or non-linear function (such as spline approach) to describe the 3D line passing between the observable parts (geometrical shapes) of the pattern.

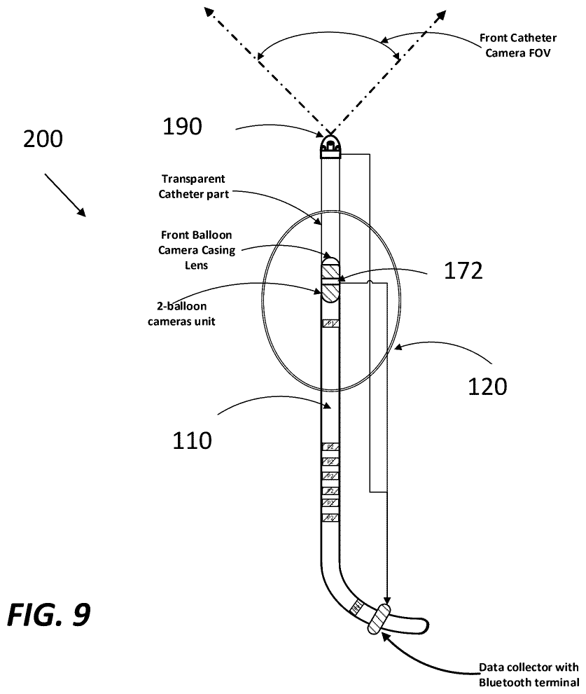

Reference is now made to FIG. 9 which is an illustration of another device 200 according to some embodiments of the invention. Device 200 of FIG. 9 may include substantially the same components and elements of device 200 of FIG. 5 and/or FIG. 6. In some embodiments, device 200 or device 100 may further include an additional image sensor 190 attached in proximity to an end of catheter 110, external to balloon 120. Image sensor 190 may be configured to follow and image the insertion process of devices 100 or 200 into the body cavity (e.g., the rectum).

In some embodiments, a testing device according to embodiments of the invention may not necessarily include a balloon.

Reference is now made to FIGS. 10A, 10B, 10C and 10D, which are illustrations of testing devices according to some embodiments of the invention.

In some embodiments, devices 300A, and 300B illustrated in FIGS. 10A and 10B may include a segmented catheter 310 comprising two or more sealed hollow segments 310A, 310B to 310N. In some embodiments, segmented catheter 310 may include 2, 3, 4, 5, 6, 7, 10 or more segments. In some embodiments, at least one of the sealed segments comprise at least one pressure sensor 130 and is connected to a neighboring segment via a flexible connector 318. In some embodiments, at least one pressure sensor 130 may be selected from polyvinylidene fluoride (PVDF) membrane, piezoresistive strain gauges, capacitive sensors, electromagnetic sensors, piezoelectric sensors, and optical sensors.

In some embodiments, each one of sealed hollow segments 310A, 310B to 310N may be filled with a fluid (e.g., a gas, liquid or gel). In some embodiments, all sealed hollow segments 310A, 310B to 310N are filled with the same type of fluid. In some embodiments, a first sealed hollow segment 310A is filled with a first type of fluid and a second hollow segment 310B is filled with a second type of fluid, different from the first. In some embodiments, sealed hollow segments 310A, 310B to 310N are configured to pivotally move one with respect to each other, via connector 318, at least around an axis perpendicular to the longitudinal axis of each sealed hollow segment 310A, 310B to 310N.

In some embodiments, devices 300A and 300B may further include one or more motion sensors 160 configured to measure a relative angle between at least two segments. 310A, 310B to 310N. In some embodiments, one or more motion sensors 160 are attached to different segments 310A, 310B to 310N. In some embodiments, one or more motion sensors 160 are accelerometers.

In the nonlimiting example illustrated in FIG. 10B, a first sealed hollow segment 310A is filled with liquid and comprises pressure sensor 130, and a second hollow segment 310B is filled with gas and comprises motion sensor 160.

In some embodiments, a device 300C may include sealed hollow segments 310A, 310B to 310N filled with a fluid (e.g., a gas, liquid or gel) and comprising two or more pressure sensors 130 located inside at least some of the sealed hollow segments. Device 300C may not include motion sensors 160. In some embodiments, pressure may be measured and monitored in different locations of the rectum, including inside the inflatable balloon 320. In this embodiment, device 300C may provide information related to the pressure versus time at different locations of the rectum in order to assist in diagnosing the patient's condition.

In some embodiments, devices 300A, 300B, and 300C may include at least one segment comprising an inflatable balloon 320 as discussed herein above, with respect to balloon 120.

In some embodiments, device 300D may include long flexible hollow segment 310, comprising a plurality of motion sensor 160. This embodiment may not include pressure sensors, therefore may provide information related to the catheter shape and of the catheter motion. Since the catheter shape follows the shape of the rectum and motion parameters reflect the activity of the rectal muscles, it may help medical professionals to diagnose the patient's condition.

Motions sensors 160, of any one of devices 300A, 300B, 300C, and 300D may be located inside at least some of segments 310A, 310B to 310N, for example, attached to flexible core 330. Flexible core 330 may be threaded through each one of segments 310A, 310B to 310N, and connectors 318. In some embodiments, at least some of motion sensors 160, of any one of devices 300A, 300B, 300C, and 300D may be located on the outer or inner surface of at least some of segments 310A, 310B to 310N.

In some embodiments, devices 300A, 300B, 300C, and 300D may further include a flexible cover 315 covering the two or more sealed hollow segments while providing a flexible connection 315 between the two or more sealed hollow segments. In some embodiments, flexible cover 315 may be made from a medical-grade elastomer.

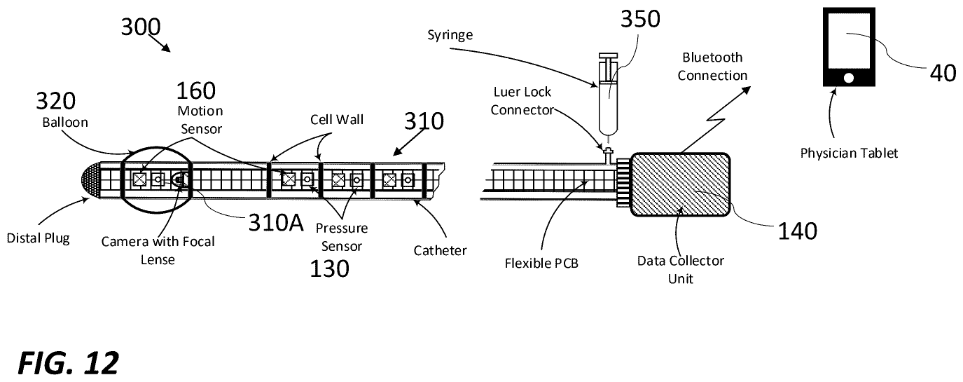

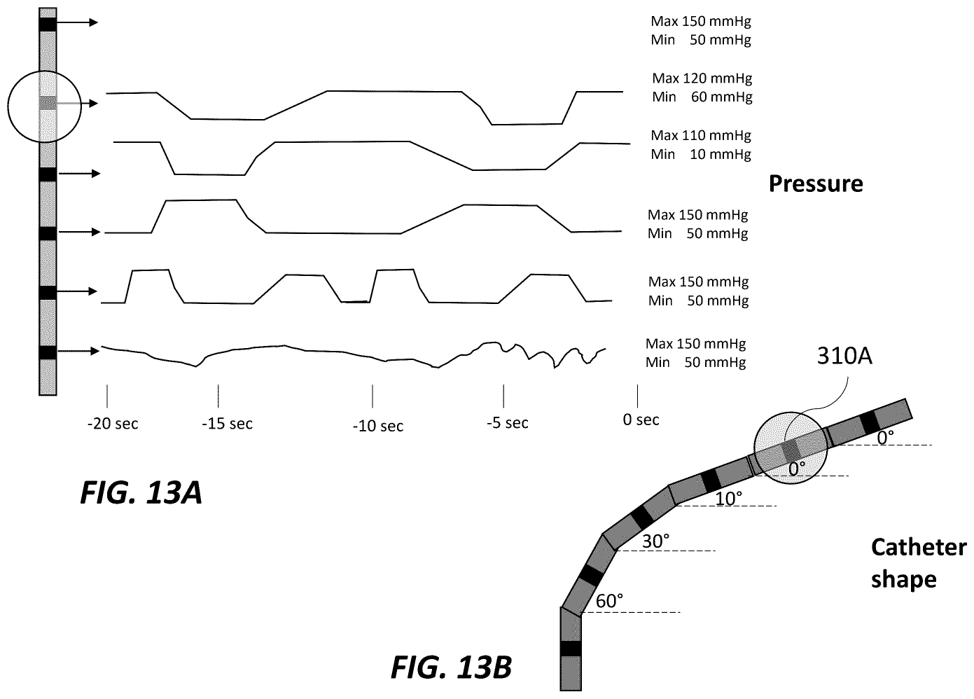

In some embodiments, devices 300A, 300B, 300C, and 300D may further include a control unit (e.g., control unit 140 illustrated in FIG. 12). In some embodiments, the control unit of devices 300A and 300B may be configured to receive pressure measurements from at least two pressure sensors 130 and the relative angle between at least two segments comprising the at least two pressure sensors; and diagnose a medical condition based on the received pressure measurements and the corresponding relative angle. A nonlimiting example for such diagnosis is given with respect to the flowchart of FIG. 17 and the discussion regarding FIGS. 13A and 13B.

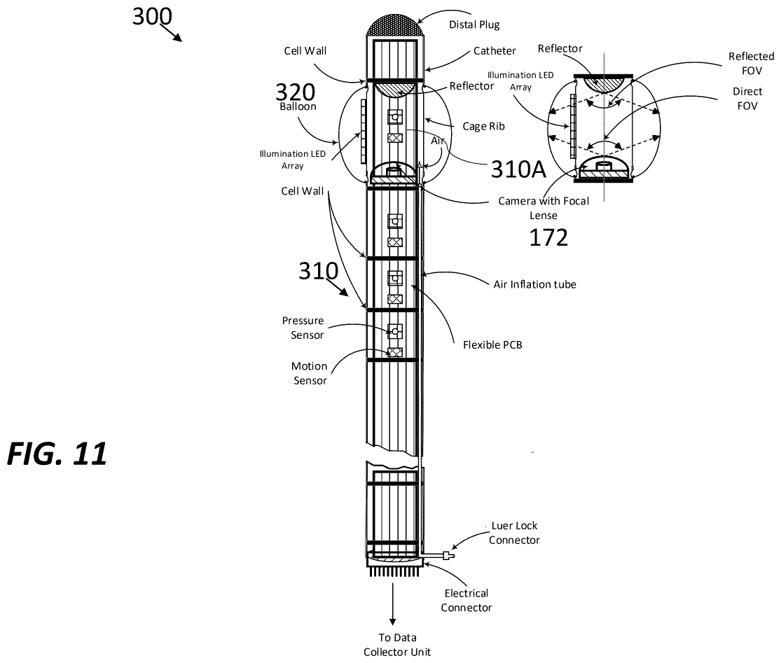

Reference is now made to FIG. 11 which is an illustration of a device 300 comprising segmented catheter 310. Device 300 may include substantially the same components as device 300A. Device 300 may further include at least one image sensor 172 embedded in one of the segments. In such a case segment 310A, is filled with gas (e.g., air) and the walls of the segments are optically transparent. In some embodiments, segment 310A further comprising a balloon 320 and image sensor 172 is configured to image at least a portion of the inner surface of the balloon as discussed herein above. In the nonlimiting example of FIG. 11 image sensor 172 is a Color CMOS LED having horizontal FOV of 95°, vertical FOV of 75°, and diagonal FOV of 113°.

Reference is now made to FIG. 12 which is an illustration of device 300 according to some embodiments of the invention. Device 300 may include substantially the same components as device 300A of FIG. 10A and device 300 of FIG. 11. Additionally, device 300 may further include a reservoir (e.g., a syringe) of fluid 350 for providing and inflating balloon 320, via an internal lumen (not illustrated) fluidically connected to balloon 320. In some embodiments, control unit 140 may receive data and measurements from motion sensors 160 and/or pressure sensors 130 and send the data and measurements to an external computing device 40, for example, via wireless communication (e.g., Bluetooth).

Reference is now made to FIGS. 13A and 13B which show results of pressure measurements from segments (FIG. 13A) and relative angles between segments measured by a device such as devices 300A and/or 300B.

Data may be collected from pressure sensors 130 and motion sensors 160. The illustration in FIG. 13B presents the shape of the catheter. Presenting shape of catheter 310 and the relative bending of each segment relative to segment 310A. The shape of the catheter may reflect the actual shape of the body cavity, for example, the patient anal channel and the rectum.

The pressure measurements may be processed prior to presenting the data to a user. FIG. 13A may be a nonlimiting example for such a presentation. The presented pressure (P_{presented}) may be calculated from the pressure reading (P_{reading}) using simple linear equation (1):

$${P}_{presented} = B + ( {P}_{reading} - {P}_{S 0} ) \cdot α \qquad ( 1 )$$

Where:
B—is the baseline, or the pressure measured by the reference sensor where no external pressure is applied.
P_{S0}—is the pressure reading by the sensor before the pressure was applied.
α—is the coefficient.

In some embodiments, control unit 140 may receive the pressure reading (P_{reading}) from pressure sensors 130 and send the measurements for further processing by computing device 40. In some embodiments, at least some of the parameters may be provided to user device 40 by a user, using a user interface. Some nonlimiting examples for such parameters may include:

$$B = 1001.14 hPa$$

$${P}_{S 0} = 1048.19 hPa$$

$$α = 1.65$$

In some embodiments, control unit 140 may receive the motion (e.g., acceleration) readings from motion sensors 130 and send the measurements for further processing by computing device 40.

In some embodiments, catheter 310 may bend mainly in one plane, therefore, catheter shape may be presented in two-dimensions, as illustrated. In some embodiments, catheter 310 may bend in space, therefore, catheter shape may be presented in three-dimensions (e.g., in perspective view).

In the nonlimiting example given in FIG. 13B the presentation of the shape of catheter 310 may include:
Bending plane may be marked on the catheter.
Catheter may be presented as a collection of straight segments.
Relative angle of each segment may be calculated based on the accelerometer output.

In some embodiments, the calculation of the catheter shape may be based on the output from motion sensors 160 (e.g., accelerometers) located in at least some of the catheter segments. Accelerometer measures force and not acceleration. Therefore, when not moving, the vector summary of all three components of the accelerator output (Aₓ, A_{y}, A_{z}) may result in g (gravity force), using equation (2).

$${A}_{x}^{2} + {A}_{y}^{2} + {A}_{z}^{2} = {g}^{2} \qquad ( 2 )$$

In some embodiments, if the result is different from g, this is an indication that the accelerometer is moving. When moving, the accelerometer output should not be used for the calculation of the accelerometer spatial orientation. For example, a threshold of +/−5% of g can be used when deciding whether the accelerometer output should be used for the calculation of the orientation.

The following non-limiting example show the various steps required for calculating the relative angle of several segments of device 300A.

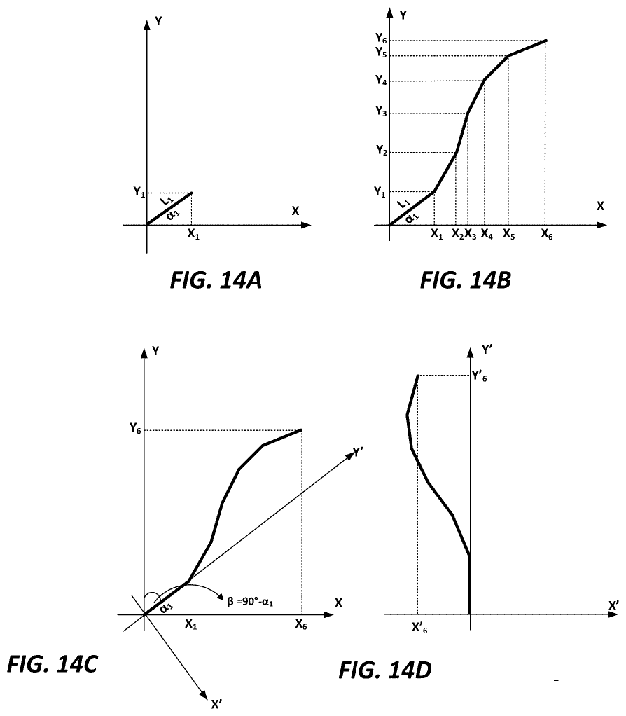

Calculation of the orientation of the first (proximal) catheter segment 310B, as illustrated in FIG. 14A. Coordinates of the first segment are calculated based on the X and Y outputs of the first accelerometer and on the known length of the first segment, using equation (3).

$$tan ( {α}_{1} ) = {A}_{Y 1} / {A}_{X 1} \qquad ( 3 )$$

$${α}_{1} = a tan ( {A}_{Y 1} / {A}_{X 1} )$$

Therefore, the coordinates of the distal end of the first segments are according to equations (4):

$${Y}_{1} = {L}_{1} * sin ( {α}_{1} ) \qquad ( 4 )$$

$${X}_{1} = {L}_{1} * cos ( {α}_{1} )$$

Similarly, the angles can be calculated for the rest of the segments, and their coordinates are illustrated in FIG. 14B.

$${Y}_{1} = {L}_{1} * sin ( {α}_{1} )$$

$${X}_{1} = {L}_{1} * cos ( {α}_{1} )$$

$${Y}_{2} = {Y}_{1} + {L}_{2} * sin ( {α}_{2} )$$

$${X}_{2} = {X}_{1} + {L}_{2} * cos ( {α}_{2} )$$

$${Y}_{3} = {Y}_{2} + {L}_{3} * sin ( {α}_{3} )$$

$${X}_{3} = {X}_{2} + {L}_{3} * cos ( {α}_{3} )$$

$${Y}_{4} = {Y}_{3} + {L}_{4} * sin ( {α}_{4} )$$

$${X}_{4} = {X}_{3} + {L}_{4} * cos ( {α}_{4} )$$

$${Y}_{5} = {Y}_{4} + {L}_{5} * sin ( {α}_{5} )$$

$${X}_{5} = {X}_{4} + {L}_{5} * cos ( {α}_{5} )$$

$${Y}_{6} = {Y}_{5} + {L}_{6} * sin ( {α}_{6} )$$

$${X}_{6} = {X}_{5} + {L}_{6} * cos ( {α}_{6} )$$

In some embodiments, the method may include transformation from Earth coordinates to the catheter coordinates, for easier presentation. Therefore, the catheter shape may be presented in catheter coordinates, rotated relative to the Earth coordinates by the angle β=90°−α₁. Catheter coordinates are marked X′-Y′, and illustrated in FIG. 14C

Instead of rotating the coordinates it is possible to rotate the representation of the catheter itself by the angle β=90°−α₁, as illustrated in FIG. 14D. For each set of coordinates (Xₙ, Yₙ) a new set (X′ₙ, Y′ₙ) may be calculated as shown below, n=1, . . . ,6.

$${R}_{n} = \sqrt{{X}_{n}^{2} + {T}_{n}^{2}}$$

$${ε}_{n} = a tan ( \frac{{Y}_{n}}{{X}_{n}} )$$

$${θ}_{n} = {ε}_{n} + β = {ε}_{n} + 90 ° - {α}_{1}$$

$${X}_{n}^{′} = {R}_{n} \cdot sin ( {θ}_{n} )$$

$${Y}_{n}^{′} = {R}_{n} \cdot cos ( {θ}_{n} )$$

Reference is now made to FIG. 15 which is a flowchart of a method of diagnosing anorectal condition according to some embodiments of the invention. The method of FIG. 15 may be performed by any processor, for example, processors included in control unit 140 and/or computing device 40, using signals received from device 100 or 100A.

In step 1510, pressure measurements may be received (optionally in real-time) from one or more pressure sensors located inside an inflatable balloon attached to a catheter, when the catheter is inserted to the rectum. For example, control unit 140 and/or computing device 40 may receive from one or more pressure sensors 130 pressure measurements. In some embodiments, control unit 140 and/or computing device 40 may create a 3D pressure map inside balloon 120 based on the pressure measurements.

In step 1520, real time pressure inside said inflatable balloon is controlled based on the measured pressure. For example, control unit 140 and/or computing device 40 may control an inflating unit in fluid 125 to inflate balloon 120 based on the pressure map. Note that in some embodiments the pressure inside the said inflatable balloon can be set manually by the medical professional. In this case the control unit will be only monitoring rather than monitoring and controlling the balloon pressure.

In some embodiments, in order to generate more accurate pressure map, pressure measurements may include receiving a plurality of pressure measurements each associated with a different section (e.g., 120A-120D) of inflatable balloon 120, such that each section forms a separate balloon volume.

In some embodiments, the method may further include receiving from at least one image sensor located inside said inflatable balloon one or more images of internal surfaces of said inflatable balloon. For example, the 3D pressure map may also be generated from one or more images of internal surfaces of said inflatable balloon. Additionally or alternatively, a 3D model of the inflatable balloon based on the images. In some embodiments, the images are received from at least two different angles, for example, from two images sensors 170 and 175.

In step 1530, pressure measurements associated with a pelvic floor disorder may be identified in the pressure measurements. In some embodiments, control unit 140 or computing device 40 may identify the pressure measurements associated with a pelvic floor disorder in real time. Additionally or alternatively, control unit 140 or computing device 40 may record the pressure measurements associated with a pelvic floor disorder to be “identified” or analyzed later, off line rather than being identified in real time.

For example, control unit 140 and/or computing device 40 may continue to control or monitor the inflation of balloon 120 until an indication may be received from the user, for example, using a user device (e.g., computing device 40) that the user senses an urge for defecation. In yet another example, control unit 140 and/or computing device 40 may continue to control or monitor the inflation of balloon 120 until an indication associated with pelvic floor disorder may be received from the user and/or a professional and/or an external computing device 40. In some embodiments, measurements associated with pelvic floor disorder, measurements associated with a sensation of urge for defecation, anal pressure level, rectal pressure level, difference between rectal and anal pressures, and the like. In some embodiments, the pressure associated with a sensation of urge for defecation is determined for each patient, for example, during several different tests.

In some embodiments, pressure measurements associated with a pelvic floor disorder may be acquired during or based on one of the following tests: balloon expulsion test, as discussed herein above, anorectal manometry, as discussed herein above, rectal sensation, tone, and compliance test (e.g., response to graded balloon distension), Neurophysiologic Tests, Pelvic MRI scan, Defecography-Radiologic Examination, Transanal Sonography, and the like.

In step 1540, anorectal condition may be diagnosed based on the identified measurements. For example, control unit 140 and/or computing device 40 may generate a pressure map of the rectum during the actual defecation process and may compare the pressure map to a previously generated map of the user stored in a database associated with device 40. In some embodiments, control unit 140 and/or computing device 40 may detect a decrease in the pressure applied by the rectal muscles indicating an anorectal condition. Additionally or alternatively, control unit 140 and/or computing device 40 may compare the real time map to a map of a healthy user stored in the database.

In some embodiments, diagnosing the anorectal condition may further be based on the received images. For example, changes in the 3D model of the rectum may be indicative of the pressure profile applied to the balloon by rectal muscles. In some embodiments, the anorectal condition is a rectocele condition and diagnosing comprises detecting distortion in the 3D model and/or the 3D pressure map indicative of budging and herniation of the rectum into the back wall of the vagina.

In some embodiments, the method may further include receiving, from one or more motion sensors attached to catheter 110, motion signals indicative of the movement of catheter 110 inside the rectum and determining an effectiveness of contraction of the rectal muscles based on the received motion signals. Determination of the effectiveness may be based, for example, on the movement pattern, such as the acceleration, the maximum velocity, frequency of the movement, or whether the movement is always in the same direction (toward the anus).

In some embodiments, following the diagnosis, control unit 140 and/or computing device 40 may control an array of electrodes located on an outer surface of the balloon to provide electrical stimulation to the rectal muscles. In some embodiments, the array may include pairs of radiofrequency (RF) electrodes configured to provide RF currents into the tissue of the rectum.

In some embodiments, the method of FIG. 15 may be conducted when catheter 110 of devices 200 and 200A is statically inserted into the rectum or while dynamically moving inside the anal and rectal canals, for example, during deflection. In some embodiments, during a dynamic test it may be beneficiary to measure the motion of catheter 110.

In some embodiments, the method of FIG. 15 may be conducted in addition or parallel to other diagnostic methods such as but not limited to: Magnetic Resonance Imaging (MRI), X-Ray imaging, and Computed Tomography (CT).

Reference is now made to FIG. 16 which is a flowchart of a method of diagnosing anorectal condition according to some embodiments of the invention. The method of FIG. 16 may be performed by any processor, for example, processors included in control unit 140 and/or computing device 40, using signals received from device 200. In step 1610, images may be received (optionally in real-time) from one or more image sensors located inside an inflatable balloon attached to a catheter, when the catheter is inserted to the rectum. For example, control unit 140 and/or computing device 40 may create a 3D model of the inflatable balloon based on the images. In some embodiments, the images are received from at least two different angles, for example, from two images sensors 170 and 175.

In step 1620, the real time pressure may be controlled inside said inflatable balloon based on the received images of internal surfaces of said inflatable balloon. For example, control unit 140 and/or computing device 40 may control an inflating unit in fluid 125 to inflate balloon 120 based on the 3D model.

In step 1630, images associated with a pelvic floor disorder may be identified. In some embodiments, control unit 140 or computing device 40 may identify the images associated with a pelvic floor disorder in real time. Additionally or alternatively, control unit 140 or computing device 40 may record the images associated with a pelvic floor disorder to be “identified” or analyzed later, off line rather than being identified in real time.

For example, control unit 140 and/or computing device 40 may continue to control the inflation of balloon 120 until an indication may be received from the user, for example, using a user device (e.g., computing device 40) that the user senses an urge for defecation. In some embodiments, 3D model of the rectum associated with a sensation of urge for defecation is determined for each patient, for example, during several different tests. In yet another example, control unit 140 and/or computing device 40 may continue to control the inflation of balloon 120 until an indication associated with a pelvic floor is received form at least one of, the user, a professional and computing device 40. In some embodiments, measurements associated with pelvic floor disorder, measurements associated with a sensation of urge for defecation, anal pressure level, rectal pressure level, difference between rectal and anal pressures, and the like.

In some embodiments, images associated with a pelvic floor disorder may be acquire during or based on one of the following tests: balloon expulsion test, as discussed herein above, anorectal manometry, as discussed herein above, rectal sensation, tone, and compliance test (e.g., response to graded balloon distension), Neurophysiologic Tests, Pelvic MRI scan, Defecography-Radiologic Examination, Transanal Sonography, and the like.

In step 1640, the anorectal condition may be diagnosed based on the identified images. For example, changes in the 3D model of the rectum may be indicative of the pressure profile applied to the balloon by rectal muscles. In some embodiments, the anorectal condition is a rectocele condition and diagnosing comprises detecting distortion in the 3D model and/or the 3D pressure map indicative of budging and herniation of the rectum into the back wall of the vagina.

In some embodiments, the method of FIG. 16 may be conducted when catheter 110 of devices 200 and 200A is statically inserted into the rectum or while dynamically moving inside the anal and rectal canals, for example, during deflection. In some embodiments, during a dynamic test it may be beneficiary to measure the motion of catheter 110.

In some embodiments, the method may further include receiving, from one or more motion sensors attached to catheter 110, motion signals indicative of the movement of catheter 110 inside the rectum and determining an effectiveness of contraction of the rectal muscles based on the received motion signals. Determination of the effectiveness may be based, for example, on the movement pattern, such as the acceleration, the maximum velocity, frequency of the movement, or whether the movement is always in the same direction (toward the anus).

In some embodiments, following the diagnosis, control unit 140 and/or computing device 40 may control an array of electrodes located on an outer surface of the balloon to provide electrical stimulation to the rectal muscles. In some embodiments, the array may include pairs of radiofrequency (RF) electrodes configured to provide RF currents into the tissue of the rectum.

In some embodiments, the method of FIG. 16 may be conducted in addition or parallel to other diagnostic methods such as but not limited to: MRI, X-Ray imaging and CT.

Reference is now made to FIG. 17 which is a flowchart of a method of diagnosing anorectal condition according to some embodiments of the invention. The method of FIG. 17 may be performed by any processor, for example, processors included in control unit 140 and/or computing device 40, using signals received from device 300, 300A, 300B, 300C and 300D. In step 1710, pressure measurements may be received (optionally in real time) from two or more pressure sensors each is attached to a segment of a segmented anorectal catheter, when the catheter is inserted to the rectum. For example, control unit 140 and/or computing device 40 may receive pressure measurements from at least two different pressure sensors 160 each being located inside a segment 310A, 310B to 310N of a segmented catheter 310. A nonlimiting example for such pressure measurements is given and discussed with respect to FIG. 13A.

In step 1720, motion measurements may be received (optionally in real-time) from motion sensors attached to at least two different segments of the segmented anorectal catheter. For example, control unit 140 and/or computing device 40 may receive motion measurements from motion sensors 160 attached to at least some of segments 310A, 310B to 310N.

In step 1730, a relative angle may be calculated between the at least two different segments based on the motion measurements. A nonlimiting example is discussed herein with respect to FIGS. 13B, and 14A-14D. In some embodiments, control unit 140 or computing device 40 may calculate the relative angle in real time. Additionally or alternatively, control unit 140 or computing device 40 may record the motion measurements to be processed at a later stage.

In step 1740, a medical condition may be diagnosed based on the received pressure measurements and a corresponding relative angle. Since the catheter shape follows the shape of the rectum, the medical professional can determine whether the shape of the rectum is normal, and use this information as an input in the diagnosis.

In some embodiments, the method of FIG. 17 may be conducted in addition or parallel to other diagnostic methods such as but not limited to: MRI, X-Ray imaging and CT.

In some embodiments, the method of FIG. 17 may be conducted when catheter 110 of devices 200 and 200A is statically inserted into the rectum or while dynamically moving inside the anal and rectal canals, for example, during deflection. In some embodiments, during a dynamic test it may be beneficiary to measure the motion of catheter 110.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A device, comprising:
a catheter;
an inflatable balloon located in a distal portion of the catheter, wherein the inflatable balloon is at least partially insertable into a body cavity;
one or more pressure sensors located inside the inflatable balloon;
an inflating unit in fluid connection with the inflatable balloon; and
a control unit attached to the catheter and configured to receive pressure measurements from the one or more pressure sensors; and to control the inflating unit to control the pressure inside the inflatable balloon based on the received measurements.

2. The device of claim 1, wherein at least one of said one or more pressure sensors is attached to a surface of said inflatable balloon.

3. The device of claim 1, wherein at least one of said one or more pressure sensors is attached to the catheter.

4. The device of claim 1, wherein the catheter is an anorectal catheter.

5. The device of claim 1, wherein said one or more pressure sensors are selected from: polyvinylidene fluoride (PVDF) membrane, piezoresistive strain gauges, capacitive sensors, electromagnetic sensors, piezoelectric sensors, and optical sensors.

6. The device of claim 1, wherein said inflatable balloon comprises two or more portions separately inflatable by a separate inlet, and wherein one or more pressure sensors are attached to a surface of each portion.

7. (canceled)

8. (canceled)

9. The device of claim 1, further comprising one or more additional pressure sensors attached to a portion of the catheter that is external to the balloon.

10. (canceled)

11. The device of claim 1, further comprising one or more image sensors located inside the balloon.

12. The device of claim 11, wherein the one or more image sensors are located at one of: in proximity to a distal end of said catheter, an inner surface of said inflatable balloon, in proximity to a distal end of the catheter and inside said catheter at a distance from said distal end.

13. (canceled)

14. (canceled)

15. The device of claim 11, wherein at least one of said image sensors is configured to capture images of the internal shape of said inflatable balloon.

16. The device of claim 1, further comprising one or more optical reflectors located inside said inflatable balloon, said one or more optical reflectors are configured to deploy upon command from said controller.

17. The device of claim 1, further comprising one or more electrodes attached on an outer surface of said inflatable balloon and configured to deliver electrical signals to rectal muscles.

18. (canceled)

19. (canceled)

20. A method of diagnosing an anorectal condition, comprising:
receiving pressure measurements from one or more pressure sensors located inside an inflatable balloon attached to a catheter, when the catheter is inserted to the rectum;
controlling in real time the pressure inside said inflatable balloon based on the real-time pressure;
identifying in said pressure measurements, measurements associated with pelvic floor disorder; and
diagnosing the anorectal condition based on the identified measurements.

21. The method of claim 20, wherein controlling the pressure inside said inflatable balloon, comprises inflating the inflatable balloon until a pressure associated with a sensation of urge for defecation is reached.

22. (canceled)

23. The method of claim 20, wherein receiving real-time pressure measurements comprises receiving a plurality of pressure measurements each associated with a different section of said inflatable balloon, and wherein each section forms a separate balloon volume.

24. The method of claim 23, wherein diagnosing the anorectal condition comprises creating a 3D pressure map inside the balloon based on the received plurality of pressure measurements.

25. The method of claim 20, further comprising:
receiving from at least one image sensor located inside said inflatable balloon one or more images of internal surfaces of said inflatable balloon; and wherein
diagnosing the anorectal condition is further based on the received images.

26. The method of claim 25, wherein diagnosing comprises constructing a 3D model of the inflatable balloon based on the images, wherein the 3D model is indicative of the pressure profile applied to the balloon by rectal muscles.

27. The method of claim 25, wherein the images are received from at least one of: two different angles inside said inflatable balloon, and at least two different image sensors located inside said inflatable balloon.

28. (canceled)

29. The method of claim 25, wherein the anorectal condition is a rectocele condition and diagnosing comprises detecting distortion in the 3D model indicative of budging and herniation of the rectum into the back wall of the vagina.

30.-50. (canceled)
